(19) United States
(12) Patent Application Publication
(10) Pub. No.: US 2019/0201924 A1
(43) Pub. Date: Jul. 4, 2019

(54) Disinfectant A pplication Apparatus And Method

(71) Applicant: Diversey, Inc., Charlotte, NC (US)

(72) Inventor: Roderick M. Dayton, Stongsville, OH (US)

(21) Appl. No.: 16/310,480

(22) PCT Filed: Jun. 26, 2017
(86) PCT No.: PCT/US17/39240

§ 371 Date: Dec. 17, 2018

Related U.S. Application Data
(60) Provisional application No. 62/354,252, filed on Jun. 24, 2016.

(51) Int. Cl. B05B 11/02; B05B 9/08; A61L 2/22; B05B 11/00

(52) U.S. Cl. CPC B05B 11/02; B05B 9/0838; A61L 9/14; B05B 11/0078; A61L 2202/15; A61L 2/22

(57) ABSTRACT

Provided are a method and apparatus for rendering a surface of an object pathogen reduced. A disinfectant compartment stores a disinfectant that exhibits an antimicrobial effect and, when applied to a surface, deactivates a portion of contagions on the surface. A neutralizer compartment stores a neutralizer that is reactive with the disinfectant to discontinue the antimicrobial effect of the disinfectant. The neutralizer is encapsulated in a shell formed of a material that interferes with a chemical reaction between the disinfectant and the neutralizer after the disinfectant and the neutralizer have been applied to the surface. A nozzle concurrently applies the disinfectant and the neutralizer onto the surface.

### BACKGROUND OF THE INVENTION

### Field of the Invention

This application relates generally to a method and apparatus for reducing contagions on an object and, more specifically, to a method and apparatus for concurrently applying a disinfectant and a neutralizing agent on an object to render the object pathogen reduced.

### 2. DESCRIPTION OF RELATED ART

Traditionally, disinfectants applied to objects in hospitals, medical offices, and other healthcare settings have been relatively low strength out of concern that a patient or other person would come into contact with the applied disinfectant. Being low strength, such disinfectants required a lengthy wet residence time on the surface of the object to effectively render that surface pathogen reduced. Cleaning personnel must monitor the applied disinfectant to ensure that it remains wet during the required residence time, which is not practical when there are many surfaces to be rendered pathogen reduced.

### BRIEF SUMMARY OF THE INVENTION

Accordingly, there is a need in the art for a method and apparatus for applying a disinfectant to a surface of an object in a manner that limits the required residence time of the disinfectant on the surface to be effective, and substantially neutralizing an active component of the disinfectant following expiration of the residence time.

According to one aspect, the subject application involves an apparatus and method for concurrently applying a disinfectant and an encapsulated neutralizer onto a surface to be rendered pathogen reduced. The neutralizer is configured to automatically neutralize the applied disinfectant after expiration of a delay following application of the neutralizer onto the surface, and/or in response to being exposed to a release stimulant.

According to another aspect, an apparatus for rendering a surface of an object pathogen reduced includes a disinfectant compartment storing a disinfectant that exhibits an antimicrobial effect and, when applied to a surface, deactivates a portion of contagions on the surface. A neutralizer compartment stores a neutralizer that is reactive with the disinfectant to discontinue the antimicrobial effect of the disinfectant. The neutralizer is encapsulated in a shell formed of a material that interferes with a chemical reaction between the disinfectant and the neutralizer after the disinfectant and the neutralizer have been applied to the surface. A nozzle concurrently applies the disinfectant and the neutralizer onto the surface.

According to another aspect, a method of rendering a surface pathogen reduced involves concurrently applying onto the surface a disinfectant and a neutralizer encapsulated in a shell. The shell interferes with a chemical reaction between the neutralizer and the disinfectant while on the surface. A chemical reaction is caused to occur between the disinfectant and the neutralizer in response to at least one of: expiration of a threshold minimum residence time of the disinfectant on the surface to achieve a desired level of pathogen reduction, and introduction of the shell to a release stimulant.

According to yet another aspect, an apparatus for rendering a surface of an object pathogen reduced includes a disinfectant compartment storing a disinfectant that exhibits an antimicrobial effect when exposed to the surface. The disinfectant is encapsulated in a shell formed of a material that isolates the disinfectant from the surface until an integrity of the shell is compromised. A nozzle directs the disinfectant onto the surface.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF SEVERAL VIEWS OF THE DRAWING

The invention may take physical form in certain parts and arrangement of parts, embodiments of which will be described in detail in this specification and illustrated in the accompanying drawings which form a part hereof and wherein:

FIG. 1 shows a side view of a disinfectant applicator and an optional mechanized receiver;

FIG. 2 shows a cross-sectional view of the disinfectant applicator taken along line 2-2 in FIG. 1; and

FIG. 3 shows and end view of an embodiment of a plunger that, when inserted into the disinfectant applicator, concurrently forces a disinfectant and a neutralizer from the disinfectant applicator onto a surface to be rendered pathogen reduced.

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

It is also to be noted that the phrase “at least one of”, if used herein, followed by a plurality of members herein means one of the members, or a combination of more than one of the members. For example, the phrase “at least one of a first widget and a second widget” means in the present application: the first widget, the second widget, or the first widget and the second widget. Likewise, “at least one of a first widget, a second widget and a third widget” means in the present application: the first widget, the second widget, the third widget, the first widget and the second widget, the first widget and the third widget, the second widget and the third widget, or the first widget and the second widget and the third widget.

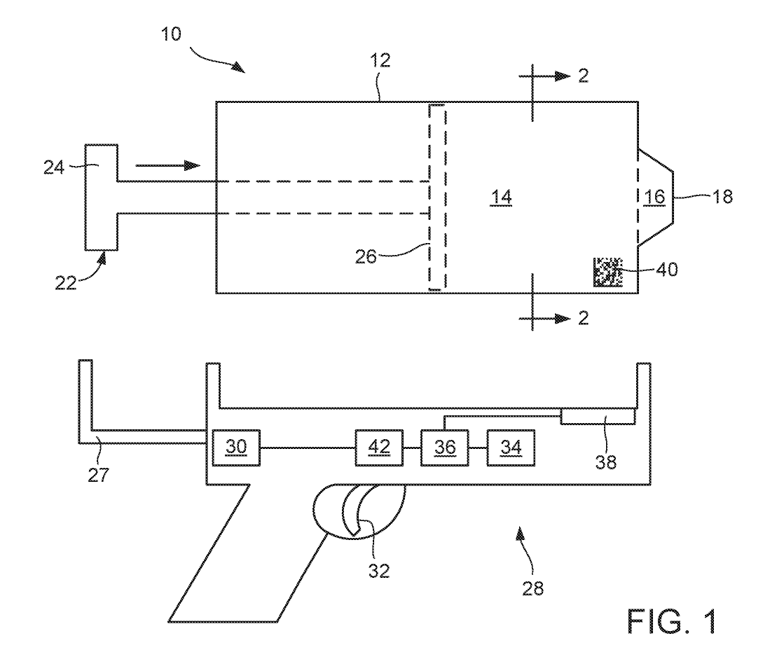

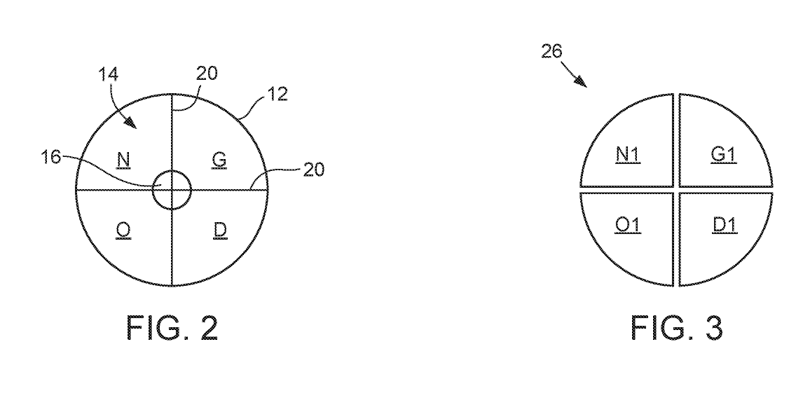

FIG. 1 provides a side view of an illustrative embodiment of a disinfectant applicator 10 storing at least a disinfectant and a neutralizer. The disinfectant applicator 10 includes a housing 12 that defines an interior cavity 14 (FIG. 2) in fluid communication with a mixing chamber 16. At least the disinfectant and the neutralizer can optionally be combined in the mixing chamber 16 prior to being sprayed from a nozzle 18 of the disinfectant applicator 10 and reaching the surface of an object to be rendered pathogen reduced. According to alternate embodiments, the disinfectant and the neutralizer can optionally be initially combined after being emitted from the disinfectant applicator 10.

The interior cavity 14 can optionally be partitioned to form a plurality of compartments, each storing a fluid isolated from another fluid stored in at least one other compartment. For example, as shown in the cross-sectional view of FIG. 2, the interior cavity 14 includes partitions 20 that collectively form four different compartments O, N, G, D, although other embodiments of the interior cavity 14 can include at least two compartments N, D, while other embodiments include at least three compartments N, D, G or N, D, O. The designations given to each compartment indicate the material or fluid stored therein.

Compartment D stores a disinfectant that, when applied to a surface for the required minimum residency time, renders the surface pathogen reduced. For example, the disinfectant can include sodium hypochlorite dissolved in water (e.g., bleach), calcium hypochlorite, etc. As another example, an alcohol-based composition (e.g., alcohol such as ethanol, isopropanol, etc., in combination with a quaternary ammonium cation). Alcohol-based disinfectants such as isopropanol-based disinfectants can achieve suitable pathogen reduction during a reasonable residence time, and leave limited amounts of residue. Aldehydes, such as formaldehyde and glutaraldehyde for example, offer sporicidal and fungicidal properties, and are at least partially deactivated by organic matter. Oxidizing agents such as electrolyzed water, hydrogen peroxide, vaporized hydrogen peroxide, accelerated hydrogen peroxide (hydrogen peroxide in combination with a surfactant or an organic acid) can be used according to embodiments of the present disclosure. Vaporized hydrogen peroxide can optionally be chosen as a disinfectant to be applied for rendering electronic devices pathogen reduced, as vaporized hydrogen peroxide decomposes to form oxygen and water, which avoids the accumulation of long-term residue. As another specific example, an aqueous solution having a performic acid concentration of up to 50%, for example, can be utilized as a disinfectant, breaking down into water and carbon dioxide.

The compartment G stores a pressurized gas such as carbon dioxide, nitrogen, etc. or other inert gas that can be stored under pressure within the compartment G, and does not readily react with the active component of the disinfectant that achieves the desired level of pathogen reduction. The gas can be used as a propellant to build pressure that aerosolizes and urges the combined materials in the mixing chamber 16 through the nozzle 18. The compartment G can be filled with a defined amount of the chosen gas to expel a majority (e.g., at least 50%, at least 70%, etc.) of the disinfectant in the compartment D without requiring the addition of more gas. Other embodiments of the disinfectant applicator 10 can include a fill port (not shown) through which the material forming the gas can be introduced into the compartment G. The fill port can optionally be in fluid communication with a pump or other source that introduces the gas used to expel the disinfectant from the disinfectant applicator 10.

The compartment O can store an optional additive, such as an optional surfactant that can be combined with the other materials in the mixing chamber 16 or after being emitted from the disinfectant applicator 10, and spray applied as part of the material combination to promote thorough wetting of the surface on which the disinfectant is to be applied. For example, the surfactant can include a wetting agent compatible with the disinfectant chosen such as dodecanoic acid, which can be used in combination with an alcohol-based composition (e.g., a combination made up of approximately 30% ethanol with dodecanoic acid). According to an alternate embodiment, the additive can include a perfume instead of, or in addition to the surfactant, that provides the combination of components applied to the surface with a pleasing or noteworthy smell to mask what may be a pungent smell of the disinfectant or simply to indicate that the surface being rendered pathogen reduced has, in fact, been sprayed with the combination of materials. According to yet another embodiment, the additive can include a material that reacts with a material encapsulating the neutralizer as described below, to cause the microencapsulating material to degrade over time to eventually expose the neutralizer to the disinfectant, and/or to promote a chemical reaction between the disinfectant and the neutralizer to limit the formation of residue or produce another product.

The compartment N stores a neutralizer that is effective to deactivate the disinfectant stored in compartment D. In other words, the neutralizer reacts with the disinfectant to discontinue the antimicrobial effect of the disinfectant. The neutralizer stored in the compartment N can be any liquid, gas, or other material that is suitable to deactivate or otherwise terminate the pathogen reduction activity of the specific disinfectant stored in the compartment D. Thus, the neutralizer can be chosen on a case-by-case basis for the specific disinfectant. However, the neutralizer can also be effective to deactivate a plurality of different disinfectants. The neutralizer chosen can be a material that is not electrically conductive to allow the combination of materials to be sprayed onto electronic devices without causing a short between electric circuit components. For example, de-ionized water, encapsulated oxygen, an organic acid, etc. can be chosen as the neutralizer. For other embodiments, the neutralizer may be electrically conductive but, in response to reacting with the disinfectant chosen, produces a substance that is not electrically conductive, and/or does not produce a reside that accumulates with each application of the disinfectant.

Regardless of the specific neutralizer chosen, the neutralizer is to be microencapsulated or otherwise adapted to exhibit its neutralizing effect on the disinfectant applied to a surface after a predetermined amount of time has passed since the combination including the disinfectant and the neutralizer was applied to the surface. For example, droplets of de-ionized water can be microencapsulated in a shell made from a material that degrades over time when exposed to air and/or the disinfectant. Thus, after such a neutralizer is sprayed from the nozzle 18 and exposed to air, or after such a neutralizer is combined with the disinfectant in the mixing chamber 16, degradation of the shell begins. The encapsulating material and/or characteristics of the shell used to encapsulate the neutralizer (e.g., shell thickness, material density, etc.) can be established as desired to create a suitable delay to allow the specific disinfectant chosen to remain active once applied to a surface for at least the required residence time to achieve the desired level of pathogen reduction. A suitable margin can also be factored into the material and/or construction of the microencapsulating shell to allow the disinfectant to remain active longer than the minimum residence time required. For example, the neutralizer can be microencapsulated to delay the neutralizing effect of the neutralizer on the disinfectant for the required residence time plus at least ten (10%) percent, fifteen (15%) percent, twenty (20%) percent, etc. Once the neutralizer begins to neutralize the applied disinfectant, the vast majority (e.g., at least seventy five (75%) percent, or at least eighty (80%) percent, or at least eighty five (85%) percent, or at least ninety (90%) percent, etc.) of the disinfectant present on the surface is deactivated in less time than would be required for the same portion of the disinfectant to be deactivated according to its ordinary active life in the absence of the neutralizer.

According to alternate embodiments, the encapsulating material forming the shell can optionally release the neutralizer in response to being exposed to a release stimulant instead of the expiration of the required residence time. For example, the shell deposited on the surface being decontaminated can release the neutralizer in response to coming into contact with a foreign object. For such embodiments, a person may come into contact with the surface to which the disinfectant and the encapsulated neutralizer have been provided. To limit the person's exposure to the disinfectant, the contact between the person and the encapsulated neutralizer can cause the shell to be broken, thereby releasing the encapsulated neutralizer and deactivating the disinfectant touched by the person.

Other embodiments, the release stimulant can include ultraviolet light, such as ultraviolet-C (“UVC”) light having a wavelength from about 100 nm to about 280 nm. Thus, the disinfectant and the encapsulated neutralizer can be applied to the surface to be rendered pathogen reduce. Following expiration of the minimum residency time required to achieve the desired level of pathogen reduction, a light that emits UVC light can be energized to release the neutralizer. For such embodiments, the residence time of the disinfectant on the surface can be established for each individual application.

According to other embodiments, the encapsulating material (and/or neutralizer) can be charged during, or prior to application onto the disinfectant. Similarly, the disinfectant can also be charged, having a polarity that the opposite of the polarity of the charged encapsulating material. For example, the encapsulating material can be charged before being inserted into the compartment N, while in the compartment N, or while being applied by the decontamination apparatus 10. The disinfectant can also be charged before being inserted into the compartment D, while in the compartment D, or while being applied by the decontamination apparatus 10. The charged encapsulating material will be attracted to the oppositely-charged disinfectant, thereby promoting uniform application of the neutralizer to the disinfectant.

Rendering the surfaces “pathogen reduced” with the decontamination apparatus 10 does not necessarily require the subject surfaces to be 100% sterile, free of any and all living organisms that can viably reproduce. Instead, to be considered pathogen reduced, there must be a lower level of living, active contagions that are viable on the decontaminated surfaces to reproduce or otherwise cause an infection after performance of the decontamination process than the level that existed on the surfaces prior to performance of the decontamination process. For example, the exposed surfaces in the bathroom can be considered to be pathogen reduced if at least a 1 log₁₀ reduction in such contagions on the surfaces (i.e., at least 90% of the contagions are deactivated, or no more than 1/10th of the biologically-active contagions originally on the exposed surfaces remain active or infectious at a time when the decontamination process is completed) occurs. According to yet other embodiments, the surfaces can be considered pathogen reduced once at least a 3 log₁₀ reduction (i.e., 1/1,000th) of such contagions on the surfaces is achieved.

The disinfectant applicator 10 can include a biasing device, an embodiment of which is shown in FIG. 1 as a plunger 22. The plunger includes a handle 24 that can optionally be manually pushed, by hand, to insert a head 26 into the housing 12 toward the mixing chamber 16. This builds a positive pressure that causes the materials stored in the plurality of compartments to be forced into the mixing chamber 16 and expelled through the nozzle 18. According to alternate embodiments, an optional mechanized receiver 28 such as that shown in FIG. 1 can receive the disinfectant applicator 10 as a replaceable cartridge. The mechanized receiver 28 includes a motor 30 or other device controlled by actuation of a trigger 32 that causes insertion of the plunger into the housing 12. For example, the motor 30 can be operable to urge an arm 27 of the mechanized receiver 28 in a direction that causes insertion of the plunger 22 into the disinfectant applicator 10 in response to actuation of the trigger 32.

To cause concurrent application of the disinfectant and the neutralizer, and in an attempt to avoid application of the disinfectant without the neutralizer, the plunger 22 can be configured to concurrently urge the material in all of the compartments present in response to the handle 24 being pushed. As shown in FIG. 3, the head 26 includes a number of subsections N1, G1, O1, D1 that are shaped to closely match the cross-section of each of the compartments N, G, O, D, respectively. Each subsection provided for the corresponding compartments provided to the housing 12 is urged into its respective compartment as a result of the handle 24 being pushed.

The above examples were described with reference to a disinfectant applied to a surface concurrently with a neutralizer. The disinfectant of such examples begins to render the surface pathogen reduced immediately upon being deposited onto the surface by the disinfectant applicator 10. Deactivation of the disinfectant would begin once the shell encapsulating the neutralizer released the neutralizer after expiration of the threshold residence time or in response to the shell being exposed to the release stimulant. However, alternate embodiments of the disinfectant can be applied to the surface encapsulated in a shell that delays pathogen reduction on the surface.

For example, the disinfectant within the chamber D can be encapsulated by a shell formed from a touch-sensitive material. The shell encapsulating the disinfectant can optionally remain intact, maintaining the disinfection in an active state in which it retains the antimicrobial effect, until a time when a person or other foreign object makes contact with the shell. Once contact occurs between the shell and the foreign object, the integrity of the shell is compromised and the disinfectant is released onto the surface on which the encapsulated disinfectant was applied.

Instead of, or in addition to being touch-sensitive, the material forming the shell of the present embodiment can optionally be ultraviolet-sensitive. An ultraviolet-sensitive shell encapsulating the disinfectant can release the encapsulated disinfectant in response to being exposed to UVC light (or light in another wavelength not commonly emitted by bulbs used to emit visible light illuminating a room). Similar to the shell formed from the touch-sensitive material, the shell formed from the ultraviolet-sensitive material remains substantially intact, isolating the disinfectant from the surface to which the encapsulated disinfectant has been applied. The disinfectant is maintained in an active state until the integrity of the shell is compromised in response to being exposed to UVC light. Regardless of the nature of the shell encapsulating the disinfectant, the encapsulated disinfectant can optionally be applied in addition to any of the additives, optionally using the propellant as described elsewhere herein.

In use, the encapsulated disinfectant can be applied to a surface to be rendered pathogen reduced. The encapsulated disinfectant can optionally be applied to the surface concurrently with application of the neutralizer. The neutralizer can optionally be encapsulated within the shell and applied as described herein concurrently with the encapsulated disinfectant. However, the neutralizer can optionally be applied without a shell concurrently with application of the encapsulated disinfectant. According to alternate embodiments, the encapsulated disinfectant can optionally be applied to the surface without a neutralizer also being applied as part of the same process. After the disinfectant is applied to the surface, the disinfectant can remain in place on the surface, available to render the surface pathogen reduced, until a time when a person or other foreign object comes into contact with the shell (touch-sensitive) or when the UVC light is applied (ultraviolet-sensitive).

The mechanized receiver 28 can also optionally be provided with a wired (e.g., Ethernet, etc. . . . ) or wireless-network (e.g., Bluetooth, WiFi, cellular, etc.) communication component 34. The degree to which the plunger 22 is inserted into the housing 12 can be determined based on operation of the motor 30 (e.g., the duration of operation, the progress of the plunger 22, etc.) by a computer-processor-based controller 36. The controller 36 can also be configured with hardware and/or software (e.g., GPS enabled antenna and software, WiFi based triangulation relative to wireless antennas, etc.) to be able to determine the location of the mechanized receiver 28 within a healthcare facility.

A usage sensor 42 can be operatively connected to the controller 36 to sense information indicative of a quantity of the disinfectant and/or neutralizer applied during a decontamination process. For example, the usage sensor 42 can include a Hall-effect circuit that can detect revolutions of a rotor provided to the motor 30, and/or a capacitive or inductive circuit that can sense an extent to which the arm 27, and accordingly the plunger 22, has been adjusted. Regardless of the sensing component of the usage sensor 42, the controller 36 can include circuitry to associate the sensed information with at least a portion of the information corresponding to the location within the healthcare facility, and/or the information obtained in response to reading a machine-readable code 40 as described below. The sensed information, optionally associated with the other information, can be transmitted over a communication network to be stored within a usage database.

Further, an optional code reader 38 can be provided to the mechanized receiver at a location where it can read or otherwise interrogate a machine-readable code (e.g., barcode, RFID tag, etc.) 40 provided to a disinfectant applicator 10 installed in the mechanized receiver 28. Such a code 40 can be used to encode a variety of information (and/or a location of a computer-readable storage medium where such information can be retrieved) including information indicative of at least one of: the identity and/or composition of at least one of the disinfectant, the neutralizer, the compressed gas and the surfactant; an expiration date of at least one of the disinfectant, the neutralizer, the compressed gas and the surfactant; and any special precautions or limitations concerning at least one of the disinfectant, the neutralizer, the compressed gas and the surfactant; and any other information relating to the disinfectant applicator 10.

Thus, during use, the controller 36 can utilize the optional code reader 38 to determine information relating to the disinfectant applicator 10 installed. The controller 36 can optionally log information documenting where the mechanized receiver 28 is used based on the location hardware and/or software, and can monitor operation of the motor 30 to estimate the extent to which the materials stored by the disinfectant applicator 10 have been consumed and optionally the quantity of such materials that remain. At least a portion, and optionally all of this information can be transmitted by the network component 34 to a data storage location external to the mechanized receiver 28 for audit purposes.

Illustrative embodiments have been described, hereinabove. It will be apparent to those skilled in the art that the above devices and methods may incorporate changes and modifications without departing from the general scope of this invention. It is intended to include all such modifications and alterations within the scope of the present invention. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An apparatus for rendering a surface of an object pathogen reduced, the apparatus comprising:
a disinfectant compartment storing a disinfectant that exhibits an antimicrobial effect and, when applied to a surface, deactivates a portion of contagions on the surface;
a neutralizer compartment storing a neutralizer that is reactive with the disinfectant to discontinue the antimicrobial effect of the disinfectant, wherein
the neutralizer is encapsulated in a shell formed of a material that interferes with a reaction between the disinfectant and the neutralizer after the disinfectant and the neutralizer have been applied to the surface; and
a nozzle that concurrently applies the disinfectant and the neutralizer onto the surface.

2. The apparatus of claim 1, wherein the neutralizer chamber is isolated from the disinfectant chamber, and the shell is structured to release the neutralizer after a threshold period of time has expired following application of the disinfectant and the neutralizer onto the surface.

3. The apparatus of claim 1, wherein the neutralizer chamber is isolated from the disinfectant chamber, and the shell is structured to release the neutralizer in response to being exposed to a release stimulant after application of the disinfectant and the neutralizer onto the surface.

4. The apparatus of claim 1 further comprising a surfactant that promotes complete coverage of the disinfectant with the neutralizer.

5. The apparatus of claim 4, wherein the surfactant is contained within a compartment that is isolated from the disinfectant compartment and the neutralizer compartment.

6. The apparatus of claim 1, wherein the disinfectant emitted from the apparatus has a first charge and the neutralizer emitted from the apparatus has a second charge that is the opposite of the first charge.

7. The apparatus of claim 1, wherein the nozzle includes a mixing chamber that combines the disinfectant with the neutralizer to form a combined spray that is emitted from the apparatus and applied to the surface to be rendered pathogen reduced.

8. The apparatus of claim 1 further comprising a scent that at least partially masks an odor of at least one of the disinfectant and the neutralizer.

9. The apparatus of claim 1 further comprising a contained propellant that causes an emission of at least one of the disinfectant and the neutralizer from the apparatus.

10. A method of rendering a surface pathogen reduced, the method comprising:
concurrently applying onto the surface:
(i) a disinfectant, and
(ii) a neutralizer encapsulated in a shell that interferes with a chemical reaction between the neutralizer and the disinfectant while on the surface; and
causing a chemical reaction to occur between the disinfectant and the neutralizer in response to at least one of:
(i) expiration of a threshold minimum residence time of the disinfectant on the surface to achieve a desired level of pathogen reduction, and
(ii) introduction of the shell to a release stimulant.

11. The method of claim 10 further comprising applying a wetting agent concurrently with application of the disinfectant and the neutralizer, wherein the wetting agent promotes complete coverage of the disinfectant with the neutralizer.

12. The method of claim 10 applying a scent concurrently with application of the disinfectant and the neutralizer, wherein the scent at least partially masks an odor of at least one of the disinfectant and the neutralizer.

13. The method of claim 10, wherein causing the chemical reaction to occur comprises exposing the shell to UVC light as the release stimulant.

14. The method of claim 10 further comprising maintaining isolation of the disinfectant from the neutralizer prior to a process of applying the disinfectant and the neutralizer onto the surface.

15. The method of claim 14 further comprising combining the disinfectant and the neutralizer as part of the process of applying the disinfectant and the neutralizer to the surface, wherein the disinfectant and the neutralizer are combined before reaching the surface.

16. An apparatus for rendering a surface of an object pathogen reduced, the apparatus comprising:
a disinfectant compartment storing a disinfectant that exhibits an antimicrobial effect when exposed to the surface, the disinfectant being encapsulated in a shell formed of a material that isolates the disinfectant from the surface until an integrity of the shell is compromised; and
a nozzle that directs the disinfectant onto the surface.

17. The apparatus of claim 16 further comprising:
a neutralizer compartment storing a neutralizer that is reactive with the disinfectant to discontinue the antimicrobial effect of the disinfectant, wherein
the neutralizer is encapsulated in a shell formed of a material that interferes with a reaction between the disinfectant and the neutralizer after the disinfectant and the neutralizer have been applied to the surface.

18. The apparatus of claim 16, wherein the shell is formed from a touch-sensitive material and the integrity of the shell is compromised in response to contact between the shell and a foreign object.

19. The apparatus of claim 16, wherein the shell is formed from an ultraviolet-sensitive material and the integrity of the shell is compromised in response to the shell being exposed to UVC light.
